(19) United States
(12) Patent Application Publication
(10) Pub. No.: US 2007/0011827 A1
(43) Pub. Date: Jan. 18, 2007

(54) Novel double para-phenylenediamines joined by a linker arm substituted with at least one group chosen from hydroxyl, alkoxy, and/or amino groups and method of dyeing keratinous fibers

(76) Inventors: Stéphane Sabelle, Paris (FR); Eric Metais, St. Leu la Foret (FR); Thierry Bordier, Tremblay en France (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/476,822

(22) Filed: Jun. 29, 2006

Related U.S. Application Data
(60) Provisional application No. 60/698,932, filed on Jul. 14, 2005.

(30) Foreign Application Priority Data
Jun. 29, 2005 (FR) 0551809

(51) Int. Cl. A61K 8/00

(52) U.S. Cl. 8/405

(57) ABSTRACT

Disclosed herein is a novel family of double para-phenylenediamines joined by a linker arm comprising at least one substituent chosen from hydroxyl, alkoxy, and/or amino substituents. Also disclosed herein is a dyeing composition comprising, in a medium suitable for dyeing, at least one para-phenylenediamine joined by a linker arm comprising at least one substituent chosen from hydroxyl, alkoxy, and/or amino substituents. Further disclosed herein is a method for dyeing keratin fibers comprising applying such a dyeing composition to the keratin fibers. A multi-compartment kit comprising at least one dyeing composition of the present disclosure and at least one oxidizing composition is also disclosed herein.

This application claims benefit of U.S. Provisional Application No. 60/698,932, filed Jul. 14, 2005, the contents of which are incorporated herein by reference. This application also claims benefit of priority under 35 U.S.C. § 119 to French Patent Application No. FR 05 51809, filed Jun. 29, 2005, the contents of which are also incorporated herein by reference.

Disclosed herein is a novel family of double para-phenylenediamines joined by a linker arm substituted with at least one group chosen from hydroxyl, alkoxy, and/or amino groups. Also disclosed herein is a dyeing composition comprising, in a medium suitable for dyeing, at least one double para-phenylenediamine joined by a linker arm substituted with at least one group chosen from hydroxyl, alkoxy, and/or amino groups. Further disclosed herein is a method for dyeing keratin fibers comprising applying such a composition to the keratin fibers.

It is known to dye keratin fibers, for example, human hair, using dye compositions comprising oxidation dye precursors, generally called oxidation bases, such as ortho- or para-phenylenediamines, ortho- or para-aminophenols, and heterocyclic compounds. These oxidation bases are colorless or weakly colored compounds which, when combined with oxidizing products, can give rise, by a process of oxidative condensation, to colored compounds.

It is also known that it is possible to vary the shades obtained with these oxidation bases by combining them with couplers or dyeing modifiers, the latter being chosen, for example, from aromatic meta-diaminobenzenes, meta-aminophenols, meta-diphenols, and certain heterocyclic compounds, such as indole compounds and pyridine compounds.

The variety of molecules available as oxidation bases and couplers makes it possible to obtain a rich palette of colors.

The “permanent” dyeing obtained using these oxidation dyes ideally satisfies at least one of a number of characteristics. For example, the dye ideally does not present any problems of a toxicological nature, makes it possible to obtain shades of the desired intensity, has good resistance to external agents, such as light, bad weather, washing, permanent waving, perspiration, and/or rubbing, provides coverage of white hair, and/or displays minimum selectivity, i.e., ensures that the smallest possible differences in coloration are obtained along the same keratin fiber, which generally is differently sensitized (i.e., damaged) between its tip and its root.

Disclosed herein are novel oxidation bases which may be capable of dyeing keratin fibers in a variety of strong, aesthetic shades with low selectivity, and which may be resistant to the various aggressive factors to which the fibers may be subjected, such as light, sweat, and/or shampoos.

Thus, disclosed herein is a novel family of double para-phenylenediamines of formula (I) and the addition salts thereof: 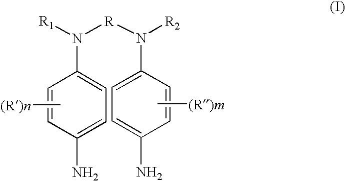
wherein:
R is chosen from:
linear or branched C₄-C₁₀ alkylene radicals substituted with at least one radical chosen from hydroxyl, C₁-C₄ alkoxy, amino, (C₁-C₄)monoalkylamino, and (C₁-C₄)dialkylamino radicals,
R₁ and R₂, which may be identical or different, are chosen from:
hydrogen,
linear or branched C₁-C₆ alkyl radicals, and
linear or branched C₁-C₆ alkyl radicals substituted with at least one radical chosen from hydroxyl, C₁-C₄ alkoxy, amino, (C₁-C₄)monoalkylamino, and (C₁-C₄)dialkylamino radicals,
R′ and R″, which may be identical or different, are chosen from:
C₁-C₆ alkyl radicals,
C₁-C₆ alkoxy radicals,
hydroxy(C₁-C₆)alkoxy radicals,
(C₁-C₆)alkoxy(C₁-C₆)alkyl radicals, and
mono- or polyhydroxy(C₁-C₆)alkyl radicals, and
n and m, which may be identical or different, are chosen from integers ranging from 0 to 4,
   with the proviso that the compound of formula (I) is not

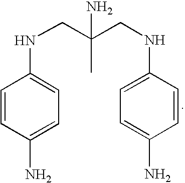

The composition of the present disclosure may make it possible to obtain a very strong coloration of keratin fibers, which may have low selectivity, and may be resistant to various external agents, for example, light.

Also disclosed herein are compositions for dyeing keratin fibers, for example, human keratin fibers, such as the hair, comprising at least one para-phenylenediamine of formula (I).

Further disclosed herein is a method for dyeing keratin fibers comprising applying a composition of the present disclosure to the keratin fibers. Still further disclosed herein is a multi-compartment device or dyeing “kit”.

Compounds

Para-phenylenediamines of Formula (I)

Non-limiting examples of para-phenylenediamines of formula (I) include, for example:

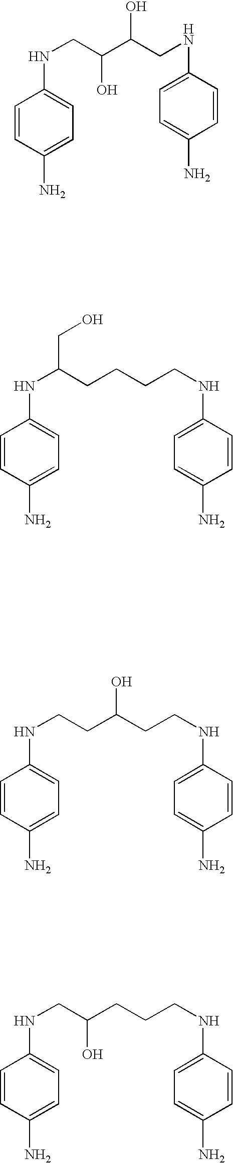

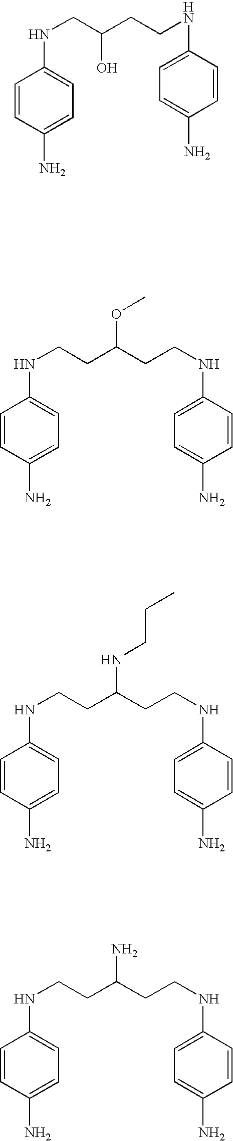

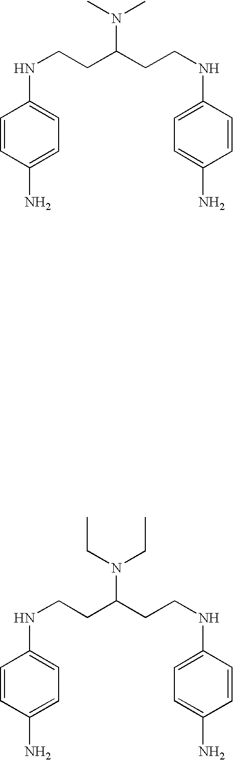

According to one embodiment, in formula (I), R₁ and R₂, which may be identical or different, are chosen from hydrogen and optionally substituted C₁-C₄ alkyl groups. In another embodiment, R may be chosen from C₄-C₆ alkylene radicals substituted with at least one radical chosen from hydroxyl, methoxy, amino, (C₁-C₄)monoalkylamino, and (C₁-C₄)dialkylamino radicals. In yet another embodiment, n and m, which may be identical or different, may be equal to 0 or 1.

The addition salts may be chosen, for example, from acid addition salts, such as hydrochloric acid, hydrobromic acid, sulphuric acid, citric acid, succinic acid, tartaric acid, lactic acid, para-toluenesulphonic acid, benzenesulphonic acid, phosphoric acid, and acetic acid.

They may also be in the form of solvates, for example, hydrates and solvates of linear or branched alcohols, such as ethanol and isopropanol.

The para-phenylenediamines of formula (I) according to the present disclosure may be prepared according to conventional methods of synthesis, for example, German Patent Application No. 101 44 226 A.

For example, the para-phenylenediamines of formula (I) may be synthesized according to the following reaction scheme:

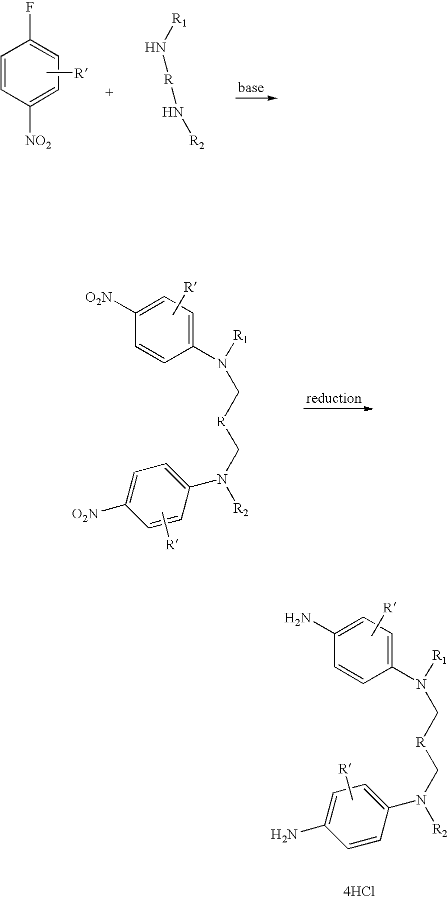

The first stage in the synthesis is a nucleophilic substitution of a diamine on a derivative of para-fluoronitrobenzene, a stage suggested, for example, by the publications Synthesis 12: 1147-1148 (1990) and Synth. Commun. 20 (22): 3537-3545 (1990). The second stage is a conventional reduction stage, and may, for example, be a hydrogenation reaction by heterogeneous catalysis in the presence of a catalyst chosen from Pd/C, Pd(II)/C, and Raney Ni, or a reduction reaction by a metal, for example, zinc, iron, tin, and the like (see, for example, J. March, Advanced Organic Chemistry, 4th ed., 1992, Wiley Interscience; M. Hudlicky, Reduction in Organic Chemistry, 1983, Ellis Honwood series Chemical Science).

Also disclosed herein are nitrogen-containing compounds of formula (II) which may be used for obtaining para-phenylenediamines of formula (I): 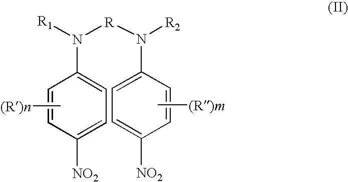
wherein R₁, R₂, R, R′, R″, n, and m are defined above in the context of formula (I), with the proviso that the compound of formula (II) is not

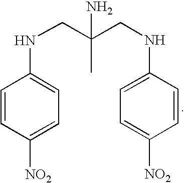

Compositions

Further disclosed herein is a dyeing composition comprising, in a medium suitable for dyeing, at least one oxidation base chosen from para-phenylenediamines of formula (I) and the addition salts thereof.

The at least one oxidation base chosen from para-phenylenediamines of formula (I) and the addition salts thereof may be present in the dye composition in an amount ranging from 0.0001 wt % to 20 wt % relative to the total weight of the dye composition, for example, from 0.01% to 10%.

Oxidation Couplers

In at least one embodiment, the composition of the present disclosure may further comprise at least one oxidation coupler.

Suitable oxidation couplers include, for example, meta-phenylenediamines, meta-aminophenols, meta-diphenols, naphthalenic couplers, heterocyclic couplers, and the addition salts thereof.

Further examples of oxidation couplers include, but are not limited to, 2-methyl-5-aminophenol, 5-N-(β-hydroxyethyl)amino-2-methylphenol, 6-chloro-2-methyl-5-aminophenol, 3-aminophenol, 1,3-dihydroxybenzene (or resorcinol), 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(β-hydroxyethylamino)toluene, and the addition salts thereof.

The at least one oxidation coupler may be present in the composition in an amount ranging from 0.0001 to 20 wt %, for example, from 0.005 to 6 wt %, relative to the total weight of the composition.

Additional Oxidation Bases

The composition according to the present disclosure may also comprise at least one additional oxidation base different from the oxidation bases of formula (I).

The at least one additional oxidation base may be chosen, for example, from para-phenylenediamines, bisphenylalkylenediamines, para-aminophenols, ortho-aminophenols, heterocyclic bases, and the addition salts thereof.

Examples of para-phenylenediamines, include, but are not limited to: para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylene-diamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxy-ethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl-β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 2-thienyl-para-phenylenediamine, 2-β-hydroxyethylamino-5-aminotoluene, 3-hydroxy-1(4′-aminophenyl)pyrrolidine, 6-(4-aminophenylamino)hexan-1-ol, and the acid addition salts thereof.

Suitable para-phenylenediamines may include, for example: para-phenylenediamine, para-toluenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-β-acetylaminoethyloxy-para-phenylenediamine, 6-(4-aminophenylamino)hexan-1-ol, and the acid addition salts thereof.

Non-limiting examples of bisphenylalkylenediamines include: N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4′-aminophenyl)tetramethylenediamine, N,N′-bis(β-hydroxyethyl)-N,N′-bis(4′-aminophenyl)tetramethylenediamine, N,N′-bis(4′-amino-3′-methylphenyl)-tetramethylenediamine, N,N′-bis(ethyl)-N,N′-bis(4′-amino-3′-methylphenyl)ethylenediamine, 1,8-bis(2,5-diaminophenxoy)-3,6-dioxaoctane, and the acid addition salts thereof.

Para-aminophenols suitable for use in accordance with the present disclosure may include, for example: para-aminophenol, 4-amino-3-methylphenol, 4-amino-3-fluorophenol, 4-amino-2-chlorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylamino-methyl)phenol, 4-amino-2-fluorophenol, 4-amino-2,6-dichlorophenol, 4-amino-6-[((5′-amino-2′-hydroxy-3′-methyl)phenyl)methyl]-2-methylphenol, bis(5′-amino-2′-hydroxy)phenylmethane, and the acid addition salts thereof.

Examples of ortho-aminophenols include, but are not limited to: 2-aminophenol, 2-amino-5-methylphenol, 2-amino-6-methylphenol, 5-acetamido-2-aminophenol, 5-[(2-hydroxyethyl)amino]-2-methylphenol, and the acid addition salts thereof.

Suitable heterocyclic bases may include, for example: pyridine derivatives, pyrimidine derivatives, and pyrazole derivatives.

Non-limiting examples of pyridine derivatives include the compounds described, for example, in British Patent Nos. 1 026 978 and 1 153 196, as well as 2,5-diaminopyridine, 2-(4-methoxyphenyl)amino-3-aminopyridine, 3,4-diaminopyridine, and the acid addition salts thereof.

Other suitable pyridine oxidation bases may include, for example, the 3-aminopyrazolo[1,5-a]pyridine oxidation bases and their acid addition salts described, for example, in French Patent Application No. 2 801 308. Examples of these compounds may include, but are not limited to: pyrazolo[1,5-a]pyridin-3-ylamine; 2-acetylaminopyrazolo[1,5-a]pyridin-3-ylamine; 2-morpholin-4-ylpyrazolo[1,5-a]pyridin-3-ylamine; 3-amino-pyrazolo[1,5-a]pyridine-2-carboxylic acid; 2-methoxypyrazolo[1,5-a]pyridin-3-ylamino; (3-aminopyrazolo[1,5-a]pyridin-7-yl)methanol; 2-(3-aminopyrazolo[1,5-a]pyridin-5-yl)ethanol; 2-(3-aminopyrazolo[1,5-a]pyridin-7-yl)ethanol; (3-aminopyrazolo[1,5-a]pyridin-2-yl)methanol; 3,6-diaminopyrazolo[1,5-a]pyridine; 3,4-diaminopyrazolo[1,5-a]pyridine; pyrazolo[1,5-a]pyridine-3,7-diamine; 7-morpholin-4-ylpyrazolo[1,5-a]pyridin-3-ylamine; pyrazolo[1,5-a]pyridine-3,5-diamine; 5-morpholin-4-ylpyrazolo[1,5-a]pyridin-3-ylamine; 2-[(3-amino-pyrazolo[1,5-a]pyridin-5-yl)-(2-hydroxyethyl)amino]ethanol; 2-[(3-aminopyrazolo[1,5-a]pyridin-7-yl)-(2-hydroxyethyl)amino]ethanol; 3-aminopyrazolo[1,5-a]pyridin-5-ol. 3-aminopyrazolo[1,5-a]pyridin-4-ol; 3-aminopyrazolo[1,5-a]pyridin-6-ol; 3-aminopyrazolo[1,5-a]pyridin-7-ol; and the acid addition salts thereof.

Pyrimidine derivatives suitable for use in accordance with the present disclosure may include, for example, the compounds described in German Patent No. 23 59 399; Japanese Patent Application No. 88-169571; Japanese Patent No. 5-63124; European Patent No. 0 77 0375; and International Patent Application No. WO 96/15765, such as 2,4,5,6-tetra-aminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, and the addition salts and tautomeric forms thereof, when a tautomeric equilibrium exists.

Examples of pyrazole derivatives may include, but are not limited to, the compounds described in German Patent Nos. 38 43 892 and 41 33 957, International Patent Application Nos. WO 94/08969 and WO 94/08970, French Patent Application No. 2 733 749, and German Patent Application No. 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazino-pyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methyl-pyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylamino-pyrazole, 3,5-diamino-4-(β-hydroxyethyl)amino-1-methylpyrazole, and the addition salts thereof.

The at least one additional oxidation base may be present in the composition in an amount ranging from 0.0001 to 20 wt %, for example, from 0.005 to 6 wt %, relative to the total weight of the composition.

Direct Dyes

The dyeing composition in accordance with the present disclosure may also comprise at least one direct dye, which may be chosen from neutral, acidic, or cationic nitrogen-containing dyes of the benzene series; neutral, acidic, or cationic direct azo dyes; neutral, acidic, or cationic quinine, for instance, anthraquinone, direct dyes; azine direct dyes; methinine direct dyes; azomethinine direct dyes; triarylmethane direct dyes, indoamine direct dyes; and natural direct dyes. In at least one embodiment, the composition according to the present disclosure may comprise at least one direct dye chosen from cationic direct dyes and natural direct dyes.

Examples of cationic direct dyes include, but are not limited to, the cationic azo direct dyes described, for instance, in International Patent Application Publication Nos. WO 95/15144 and WO 95/01772 and European Patent No. 0 714 954.

These compounds may include, for example:
1,3-dimethyl-2-[[4-(dimethylamino)phenyl]azo]-1H-imidazolium chloride,
1,3-dimethyl-2-[(4-aminophenyl)azo]-1H-imidazolium chloride, and
1-methyl-4-[(methylphenylhydrazono)methyl]pyridinium methyl sulphate.

Suitable natural direct dyes may include, for example: lawsone, juglone, alizarin, purpurin, carminic acid, kermesic acid, purpurogallin, protocatechaldehyde, indigo, isatin, curcumine, spinulosine, and apigenidine. In one embodiment, extracts or decoctions comprising these natural dyes may also be used, for instance, henna-based cataplasms or extracts.

The at least one direct dye may be present in the composition in an amount ranging from 0.001 to 20 wt % of the total weight of the composition, for example, from 0.005 to 10 wt %.

Dyeing Medium

The medium suitable for dyeing may be chosen from water and mixtures of water and at least one organic solvent, for example, linear or branched C₁-C₄ lower alcohols, such as ethanol and isopropanol; polyols and polyol ethers such as 2-butoxy-ethanol, propylene glycol, propylene glycol monomethyl ether, diethylene glycol monoethyl ether, diethylene glycol monomethyl ether, and glycerol; aromatic alcohols such as benzyl alcohol and phenoxyethanol; and mixtures thereof.

The at least one solvent may be present in the dyeing composition in an amount ranging from 1 to 40 wt % relative to the total weight of the dyeing composition, for example, from 5 to 30 wt %.

Cosmetic Adjuvants

In at least one embodiment, the dyeing composition may comprise at least one cosmetic adjuvant chosen from antioxidants, penetrants, sequestering agents, perfumes, buffers, dispersants, surfactants, conditioners, film-forming agents, polymers, ceramides, preservatives, lustre agents, opacifiers, vitamins, and provitamins.

The at least one cosmetic adjuvant may be present in the composition in an amount, for each of them, ranging from 0.01 to 20 wt % relative to the weight of the composition.

The pH of the composition of the present disclosure may range from 3 to 12, for example, from 5 to 11. The pH may be adjusted to a desired value by means of acidifying or basifying agents conventionally used in the dyeing of keratin fibers, or by means of conventional buffer systems.

Non-limiting examples of acidifying agents include: inorganic or organic acids other than dicarboxylic acids, such as hydrochloric acid, orthophosphoric acid, and sulphuric acid; carboxylic acids, such as acetic acid, tartaric acid, citric acid, and lactic acid; and sulphonic acids.

Suitable basifying agents may include, for example: aqueous ammonia, alkali metal carbonates, alkanolamines such as mono-, di-, and triethanolamines and their derivatives, sodium hydroxide, potassium hydroxide, and compounds of formula (III): 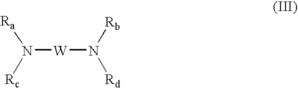
wherein:

W is chosen from propylene residues optionally substituted with at least one entity chosen from hydroxyl groups and C₁-C₄ alkyl radicals; and

Rₐ, R_{b}, R_{c}, and R_{d}, which may be identical or different, are chosen from hydrogen, C₁-C₄ alkyl radicals, and C₁-C₄ hydroxyalkyl radicals.

It is to be understood that a person skilled in the art will ensure that the envisioned optional additions, for example, the addition of at least one adjuvant, at least one additional oxidation dye precursor, at least one oxidation coupler, and/or at least one direct dye, are chosen such that the advantageous properties intrinsically associated with the oxidation dyeing composition in accordance with the present disclosure are not, or are not substantially, adversely affected.

The dyeing composition according to the present disclosure may be in various forms, such as liquids, creams, and gels, or any other form suitable for dyeing keratin fibers, for example, human hair.

Method

Also disclosed herein is a method for dyeing keratin fibers comprising applying a composition of the present disclosure to the keratin fibers, and developing the color using at least one oxidizing agent. The color may be developed at acidic, neutral, or alkaline pH. In one embodiment, the oxidizing agent may be added to the composition of the present disclosure at the moment of use. According to another embodiment, the oxidizing agent can be added in an oxidizing composition comprising it, which may be applied simultaneously with or sequentially to the composition of the present disclosure.

Examples of oxidizing agents include, but are not limited to, hydrogen peroxide; urea peroxide; alkali metal bromates; persalts, such as perborates and persulphates; peracids; and oxydase enzymes, such as peroxydases, 2-electron oxydoreductases, such as uricases, and 4-electron oxygenases, such as laccases. In at least one embodiment, the at least one oxidizing agent is hydrogen peroxide.

According to another embodiment, the composition according to the present disclosure may be mixed, for example, at the moment of use, with a composition comprising, in a medium suitable for dyeing, at least one oxidizing agent, this oxidizing agent being present in a sufficient amount for developing a desired coloration. The mixture obtained may then be applied to the keratin fibers. After a holding time ranging from 3 to 50 minutes, for example, from 5 to 30 minutes, the keratin fibers may be rinsed, washed with shampoo, rinsed again, and then dried.

The oxidizing composition may also comprise various adjuvants conventionally used in compositions for hair dyeing and as defined above.

The pH of the oxidizing composition comprising the oxidizing agent may be such that, after mixing with the dyeing composition, the pH of the resulting composition applied to the keratin fibers ranges from 3 to 12, for example, from 5 to 11. The pH may be adjusted to a desired value by means of acidifying or basifying agents conventionally used in the dyeing of keratin fibers and as defined above.

The ready-to-use composition which is finally applied to the keratin fibers may be in various forms, such as liquids, creams, and gels, or any other form suitable for dyeing keratin fibers, for example, human keratin fibers, such as the hair.

Multi-Compartment Device

Further disclosed herein is a multicompartment device or dyeing “kit” comprising at least one first compartment containing at least one dyeing composition comprising at least one oxidation base chosen from para-phenylenediamines of formula (I) and the addition salts thereof, and at last one second compartment containing at least one oxidizing composition. This device may be equipped with a means for delivering the desired mixture onto the hair, such as the devices described in French Patent No. 2 586 913.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below.

### EXAMPLES

### Examples of Synthesis

### Example 1

### Synthesis of 1,4-bis[(4-aminophenyl)amino]butane-2,3-diol tetrahydrochloride (2)

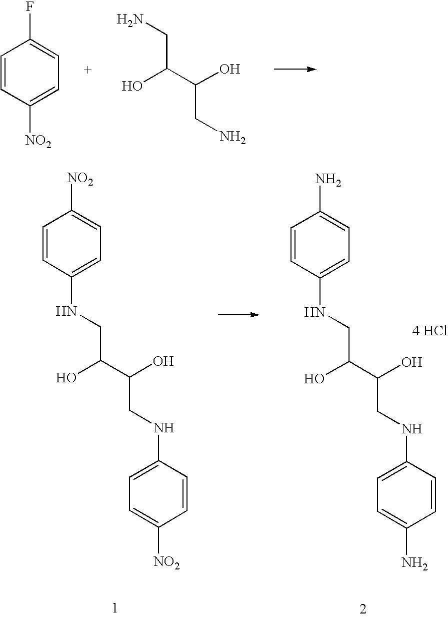 Stage 1: Synthesis of 1,4-bis[(4-nitrophenyl)amino]butane-2,3-diol (1)

3.65 g of 4-fluoronitrobenzene (2 eq.) were dissolved in 100 ml of DMSO. 1.2 equivalents of meso-1,4-diamino-2,3-butanediol and 6 equivalents of triethylamine were added to the solution. The reaction medium was brought to 80° C. for 20 hours. The mixture was then poured onto crushed ice, and a precipitate formed. The latter was filtered off, washed with water, and then dried.

Stage 2: Synthesis of 1,4-bis[(4-aminophenyl)amino]butane-2,3-diol tetrahydrochloride (2)

The nitrated compound was solubilized in 500 ml of ethanol, in a 1 L hydrogenator. 10% palladium-on-charcoal (50% water content) was added and the hydrogenator was charged with hydrogen. After reaction for 1 h 45 min, the palladium was filtered off and 20 ml of 3M hydrochloric ethanol and 300 ml of isopropyl ether were added to the filtrate. The precipitate obtained was filtered and then recrystallized from hydrochloric ethanol.

The mass and proton NMR spectra were in accordance with the expected structure of the product.

### Example 2

### Synthesis of 2,6-bis[(4-aminophenyl)amino]hexan-1-ol tetrahydrochloride (5)

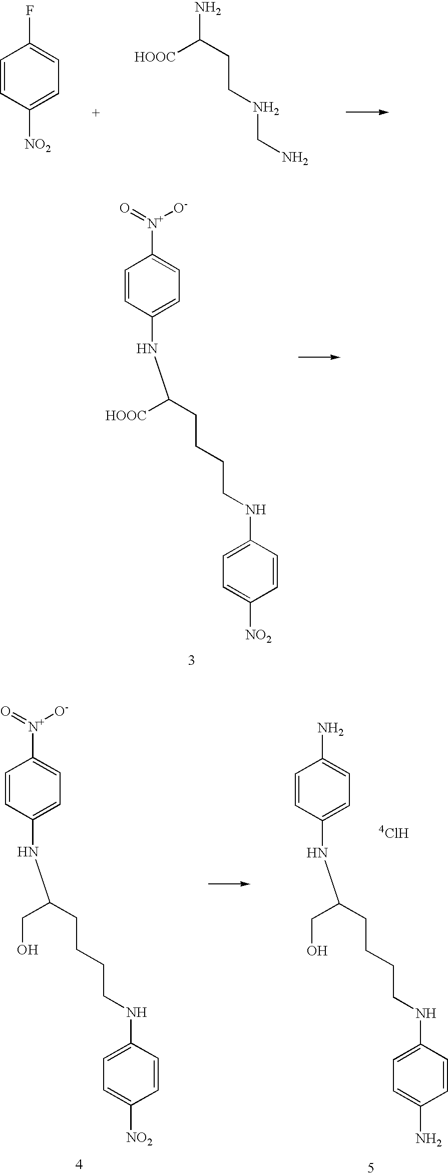 Stage 1: Synthesis of 2,6-bis[(4-nitrophenyl)amino]hexanoic acid (2)

5 g (34.2 mmol) of L-lysine were dissolved in 100 ml of water in the presence of 1.4 g (1 eq.) of sodium hydroxide and 8.6 g (3 eq.) of sodium bicarbonate, in a 250 ml three-necked flask equipped with a condenser and a thermometer. A solution of 10.8 ml (3 eq.) of 4-fluoronitrobenzene in 60 ml of ethanol was poured onto the mixture, which was brought to reflux (85°-90° C.) for 5 days. The cooled mixture was extracted with ethyl ether. The aqueous phase was acidified to pH˜3 with 5 N hydrochloric acid. A gum-like precipitate formed and was extracted with dichloromethane in the presence of a small amount of methanol. The organic phase was washed with water until neutrality was reached, dried over sodium sulphate, and then evaporated to give an orange-colored oil which crystallized in the presence of ethyl ether. 11.7 g of a yellow crystalline product were obtained, i.e., an 88% yield.

Stage 2: Synthesis of 2,6-bis[(4-nitrophenyl)amino]hexan-1-ol (4)

29 g of 2,6-bis[(4-nitrophenyl)amino]hexanoic acid (3) and 290 ml of THF were introduced into a 1 L reactor while flushing with nitrogen.

3 equivalents of borane were added by means of a dropping funnel. The mixture was left to stir for 12 hours at 20° C. and then cooled to 15° C. and methanol (100 ml) was run in dropwise. 1 hour later, the mixture was evaporated to dryness. 100 ml of water were added with stirring and then the solid formed was filtered off. This solid was recrystallized with 100 ml of isopropanol.

Stage 3: Synthesis of 2,6-bis[(4-aminophenyl)amino]hexan-1-ol tetrahydrochloride (5)

The nitrated compound (4) was solubilized in 250 ml of methanol in a 1 L hydrogenator. 10% palladium-on-charcoal (50% water content) was added and the hydrogenator was loaded with hydrogen. After reaction for 1 h 30 min, the palladium was filtered off. The reaction medium was poured into a mixture of 30 ml of HCl and 200 ml of isopropanol. The precipitate obtained was filtered off and recrystallized from hydrochloric ethanol.

The mass and proton NMR spectra were in accordance with the expected structure of the product.

### Example 3

### Synthesis of 1,5-bis(4-aminophenylamino)pentan-3-ol 8

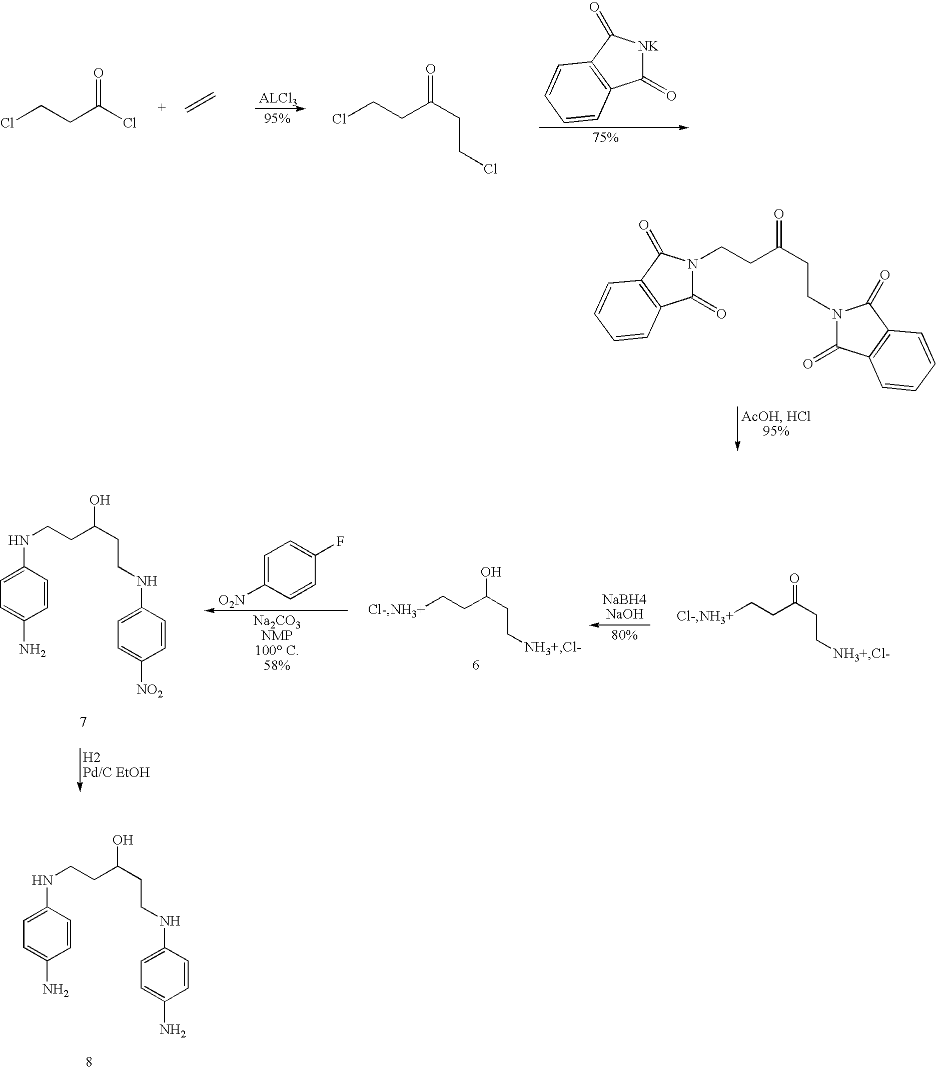 Stage 1: Synthesis of 1,5-diaminopentan-3-ol dihydrochloride 6:

The 1,5-diaminopentan-3-ol 6 was synthesized in 4 stages from ethylene and 3-chloropropionyl chloride with an overall yield of 54% according to a reaction scheme described in the references below:
Bull. Chem. Soc. Jpn., 55: 2404-2408 (1982);
Bull. Chem. Soc. Jpn., 65: 334-339 (1992);
Bull. Chem. Soc. Jpn., 75: 2595-2607 (2002);
Recl. Trav. Chim. Pays-Bas, 112: 535-548 (1993);
J. Chem. Soc. (C) Organic 17: 2401-2403 (1976); and
European Patent Application No. 0 872 466 A1.
   Stage 2: Synthesis of 1,5-bis(4-nitrophenylamino)-pentan-3-ol 7:

30.33 g of sodium carbonate (4 eq.) and 6.77 g of 1,5-diaminopentan-3-ol (0.5 eq.) were added to a solution of 10 g of 4-fluoronitrobenzene (1 eq.) in 100 ml of NMP. The resulting reaction medium was heated at 100° C. for 5 days. After a return to ambient temperature, the medium was poured over crushed ice. The precipitate obtained was filtered off, washed with water, dried, and then purified by chromatography on a silica column (eluent: 25/75 EtOAc/DCM). 14.8 g of expected product were obtained with a 58% yield.

Stage 3: Synthesis of 1,5-bis(4-aminophenylamino)pentan-3-ol 8:

2.5 g of 1,5-bis(4-nitrophenylamino)pentan-3-ol 7 and 0.5 g of palladium-on-charcoal (at 5% (50% water content)) were added to 250 ml of absolute ethanol in a 300 ml hydrogenator. The reactor was placed under a hydrogen pressure of 20 bar at a temperature of 50° C. for 2 h 10 min. The reaction medium was then filtered and poured into a hydrochloric isopropanol solution. The precipitate formed was filtered off, rinsed with isopropyl ether, and then dried. 1.98 g of 1,5-bis(4-aminophenylamino)pentan-3-ol 8 were thus obtained, with a 76% yield, in the form of a white powder.

The mass and proton NMR spectra were in accordance with the expected structure of the product.

### Examples of Dyeing

### Examples 1 to 13

### Dyeing Composition Based on 1,4-bis[(4-aminophenyl)amino]butane-2,3-diol tetrahydrochloride (2)

### Examples 1 to 7

### Dyeing in an Acid Medium

The following dyeing compositions were prepared:

| | Example | | | | | | |
|---|---|---|---|---|---|---|---|
| | 1 | 2 | 3 | 4 | 5 | 6 | 7 |
| 1,4-bis[(4-Aminophenyl)amino]- | 10⁻³ mol | 10⁻³ mol | 10⁻³ mol | 10⁻³ mol | 10⁻³ mol | 10⁻³ mol | 10⁻³ mol |
| butane-2,3-diol tetrahydrochloride (2) | | | | | | | |
| Benzene-1,3-diol | 10⁻³ mol | | | | | | |
| 5-Amino-2-methylphenol | | 10⁻³ mol | | | | | |
| 1H-Indol-6-ol | | | 10⁻³ mol | | | | |
| 2-Aminopyridin-3-ol | | | | 10⁻³ mol | | | |
| 3,6-Dimethyl-1H-pyrazolo[5,1-c]- | | | | | 10⁻³ mol | | |
| [1,2,4]triazole | | | | | | | |
| 2-(2,4-Diaminophenoxy)ethanol | | | | | | 10⁻³ mol | |
| hydrochloride | | | | | | | |
| 3-Amino-2-chloro-6-methylphenol | | | | | | | 10⁻³ mol |
| hydrochloride | | | | | | | |
| Dye support (1) | (*) | (*) | (*) | (*) | (*) | (*) | (*) |

(*): Dye support (1) pH 7

| | | |
|---|---|---|
| 96° Ethyl alcohol | 20.8 | g |
| Sodium metabisulphite, 35% aqueous solution | 0.23 | g A.M |
| Pentasodium salt of diethylenetriaminepentaacetic | 0.48 | g A.M |
| acid, 40% aqueous solution | | |
| C₈-C₁₀ Alkyl polyglucoside, 60% aqueous solution | 3.6 | g A.M |
| Benzyl alcohol | 2.0 | g |
| Polyethylene glycol with 8 ethylene oxide units | 3.0 | g |
| Na₂HPO₄ | 0.28 | g |
| KH₂PO₄ | 0.46 | g |

At the moment of use, each composition was mixed with an equal weight of aqueous hydrogen peroxide at 20 volumes (6 wt %). A final pH of 7 was obtained.

Each mixture obtained was applied to locks of grey hair comprising 90% white hairs. After a holding time of 30 minutes, the locks were rinsed, washed with a standard shampoo, rinsed again, and then dried.

The shades obtained are shown in the table below:

| | Example | | | | | | |
|---|---|---|---|---|---|---|---|
| | 1 | 2 | 3 | 4 | 5 | 6 | 7 |
| Shade | orangey | deep | deep | deep | deep | deep | deep |
| observed | brown | grey- | grey | red- | red | blue | violet- |
| | | violet- | | brown | | | blue |
| | | red | | | | | |

### Examples 8 to 13

### Dyeing in a Basic Medium

The following dyeing compositions are prepared:

| | Example | | | | | |
|---|---|---|---|---|---|---|
| | 8 | 9 | 10 | 11 | 12 | 13 |
| 1,4-bis[(4-Aminophenyl)amino]butane- | 10⁻³ mol | 10⁻³ mol | 10⁻³ mol | 10⁻³ mol | 10⁻³ mol | 10⁻³ mol |
| 2,3-diol tetrahydrochloride (2) | | | | | | |
| Benzene-1,3-diol | 10⁻³ mol | | | | | |
| 5-Amino-2-methylphenol | | 10⁻³ mol | | | | |
| 1 H-Indol-6-ol | | | 10⁻³ mol | | | |
| 2-Aminopyridin-3-ol | | | | 10⁻³ mol | | |
| 2-(2,4-Diaminophenoxy)ethanol | | | | | 10⁻³ mol | |
| hydrochloride | | | | | | |
| 3-Amino-2-chloro-6-methylphenol | | | | | | 10⁻³ mol |
| hydrochloride | | | | | | |
| Dye support (2) | (*) | (*) | (*) | (*) | (*) | (*) |
| Demineralized water q.s. | 100 g | 100 g | 100 g | 100 g | 100 g | 100 g |

(*): Dye support (2) pH 9.5

| | | |
|---|---|---|
| 96° Ethyl alcohol | 20.8 | g |
| Sodium metabisulphite, 35% aqueous solution | 0.23 | g A.M |
| Pentasodium salt of diethylenetriaminepentaacetic | 0.48 | g A.M |
| acid, 40% aqueous solution | | |
| C₈-C₁₀ Alkyl polyglucoside, 60% aqueous solution | 3.6 | g A.M |
| Benzyl alcohol | 2.0 | g |
| Polyethylene glycol with 8 ethylene oxide units | 3.0 | g |
| NH₄Cl | 4.32 | g |
| Aqueous ammonia containing 20% of NH₃ | 2.94 | g |

At the moment of use, each composition was mixed with an equal weight of aqueous hydrogen peroxide at 20 volumes (6 wt %). A final pH of 9.5 was obtained.

Each mixture obtained was applied to locks of grey hair comprising 90% white hairs. After a holding time of 30 minutes, the locks were rinsed, washed with a standard shampoo, rinsed again, and then dried.

The shades obtained are shown in the table below:

| | Example | | | | | |
|---|---|---|---|---|---|---|
| | 8 | 9 | 10 | 11 | 12 | 13 |
| Shade | orangey | deep | orangey | orangey | deep | deep grey- |
| observed | | violet- | | | blue | violet |
| | | red | | | | |

What is claimed is:

1. A para-phenylenediamine compound chosen from compounds of formula (I) and the addition salts thereof: 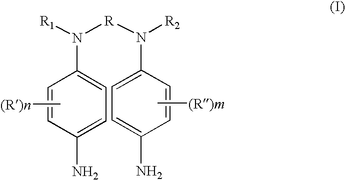
wherein:
R is chosen from:
linear or branched C₄-C₁₀ alkylene radicals substituted with at least one radical chosen from hydroxyl, C₁-C₄ alkoxy, amino, (C₁-C₄)monoalkylamino, and (C₁-C₄)dialkylamino radicals,
R₁ and R₂, which may be identical or different, are chosen from:
hydrogen,
linear or branched C₁-C₆ alkyl radicals, and
linear or branched C₁-C₆ alkyl radicals substituted with at least one radical chosen from hydroxyl, C₁-C₄ alkoxy, amino, (C₁-C₄)monoalkylamino, and (C₁-C₄)dialkylamino radicals,
R′ and R″, which may be identical or different, are chosen from:
C₁-C₆ alkyl radicals,
C₁-C₆ alkoxy radicals,
hydroxy(C₁-C₆)alkoxy radicals,
(C₁-C₆)alkoxy(C₁-C₆)alkyl radicals, and
mono- or polyhydroxy(C₁-C₆)alkyl radicals, and
n and m, which may be identical or different, are chosen from integers ranging from 0 to 4,
with the proviso that the para-phenylenediamine compound is not:

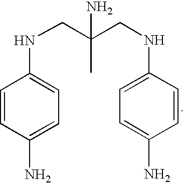

2. The para-phenylenediamine compound of claim 1, wherein R is chosen from C₄-C₆ alkylene radicals substituted with at least one radical chosen from hydroxyl, methoxy, amino, (C₁-C₄)alkylamino, and (C₁-C₄)dialkylamino radicals.

3. The para-phenylenediamine compound of claim 1, wherein R is chosen from linear or branched C₄-C₆ alkylene radicals substituted with at least one hydroxyl radical.

4. The para-phenylenediamine compound of claim 1, wherein R₁ and R₂, which may be identical or different, are chosen from hydrogen and optionally substituted C₁-C₄ alkyl groups.

5. The para-phenylenediamine compound of claim 1, wherein n and m, which may be identical or different, are equal to 0 or 1.

6. The para-phenylenediamine compound of claim 1, wherein the addition salts are chosen from hydrochloric acid, hydrobromic acid, sulphuric acid, citric acid, succinic acid, tartaric acid, lactic acid, para-toluenesulphonic acid, benzenesulphonic acid, phosphoric acid, and acetic acid addition salts, it being possible for these compounds to be optionally in the form of solvates.

7. The para-phenylenediamine compound of claim 1, chosen from:

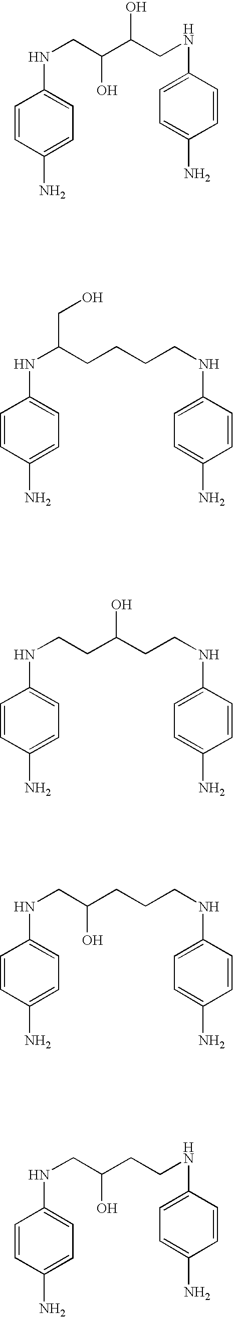

8. The para-phenylenediamine compound of claim 7, chosen from:

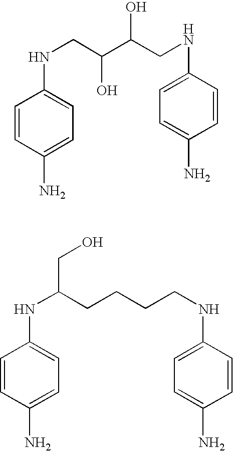

9. A dyeing composition comprising, in a medium suitable for dyeing, at least one para-phenylenediamine oxidation base chosen from compounds of formula (I) and the addition salts thereof: 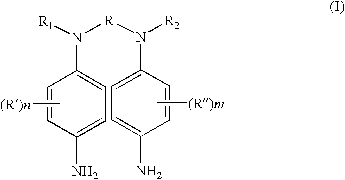
wherein:
R is chosen from:
linear or branched C₄-C₁₀ alkylene radicals substituted with at least one radical chosen from hydroxyl, C₁-C₄ alkoxy, amino, (C₁-C₄)monoalkylamino, and (C₁-C₄)dialkylamino radicals,
R₁ and R₂, which may be identical or different, are chosen from:
hydrogen,
linear or branched C₁-C₆ alkyl radicals, and
linear or branched C₁-C₆ alkyl radicals substituted with at least one radical chosen from hydroxyl, C₁-C₄ alkoxy, amino, (C₁-C₄)monoalkylamino, and (C₁-C₄)dialkylamino radicals,
R′ and R″, which may be identical or different, are chosen from:
C₁-C₆ alkyl radicals,
C₁-C₆ alkoxy radicals,
hydroxy(C₁-C₆)alkoxy radicals,
(C₁-C₆)alkoxy(C₁-C₆)alkyl radicals, and
mono- or polyhydroxy(C₁-C₆)alkyl radicals, and
n and m, which may be identical or different, are chosen from integers ranging from 0 to 4,
with the proviso that the at least one para-phenylenediamine oxidation base is not:

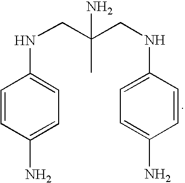

10. The composition of claim 9, wherein the at least one para-phenylenediamine oxidation base is present in the composition in an amount ranging from 0.0001 wt % to 20 wt % relative to the total weight of the composition

11. The composition of claim 10, wherein the at least one para-phenylenediamine oxidation base is present in the composition in an amount ranging from 0.01 wt. % to 10 wt. % relative to the total weight of the composition.

12. The composition of claim 9, further comprising at least one oxidation coupler.

13. The composition of claim 12, wherein the at least one oxidation coupler is chosen from meta-phenylenediamines, meta-aminophenols, meta-diphenols, naphthalenic couplers, heterocyclic couplers, and the addition salts thereof.

14. The composition of claim 9, further comprising at least one additional oxidation base different from the oxidation bases of formula (I).

15. The composition of claim 14, werein the at least one additional oxidation base is chosen from para-phenylenediamines, bisphenylalkylenediamines, para-aminophenols, ortho-aminophenols, heterocyclic bases, and the addition salts thereof.

16. The composition of claim 9, further comprising at least one direct dye.

17. The composition of claim 9, wherein the medium suitable for dyeing is chosen from water and mixtures of water and at least one organic solvent.

18. The composition of claim 17, wherein the at least one organic solvent is chosen from linear or branched C₁-C₄ lower alcohols, aromatic alcohols, and mixtures thereof.

19. The composition of claim 9, further comprising at least one cosmetic adjuvant chosen from antioxidants, penetrants, sequestering agents, perfumes, buffers, dispersants, surfactants, conditioners, film-forming agents, polymers, ceramides, preservatives, lustre agents, opacifiers, vitamins, and provitamins.

20. The composition of claim 19, wherein the at least one cosmetic adjuvant is present in the composition in an amount, for each of them, ranging from 0.01 to 20 wt % relative to the total weight of the composition.

21. The composition of claim 1, further comprising at least one oxidizing agent.

22. A method for dyeing keratin fibers, comprising applying at least one dyeing composition to the keratin fibers in the presence of at least one oxidizing agent for a time sufficient to develop a desired coloration,
wherein the at least one dyeing composition comprises, in a medium suitable for dyeing, at least one para-phenylenediamine oxidation base chosen from compounds of formula (I) and the addition salts thereof: 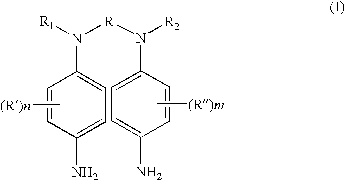
wherein:
R is chosen from:
linear or branched C₄-C₁₀ alkylene radicals substituted with at least one radical chosen from hydroxyl, C₁-C₄ alkoxy, amino, (C₁-C₄)monoalkylamino, and (C₁-C₄)dialkylamino radicals,
R₁ and R₂, which may be identical or different, are chosen from:
hydrogen,
linear or branched C₁-C₆ alkyl radicals, and
linear or branched C₁-C₆ alkyl radicals substituted with at least one radical chosen from hydroxyl, C₁-C₄ alkoxy, amino, (C₁-C₄)monoalkylamino, and (C₁-C₄)dialkylamino radicals,
R′ and R″, which may be identical or different, are chosen from:
C₁-C₆ alkyl radicals,
C₁-C₆ alkoxy radicals,
hydroxy(C₁-C₆)alkoxy radicals,
(C₁-C₆)alkoxy(C₁-C₆)alkyl radicals, and
mono- or polyhydroxy(C₁-C₆)alkyl radicals, and
n and m, which may be identical or different, are chosen from integers ranging from 0 to 4,
with the proviso that the at least one para-phenylenediamine oxidation base is not:

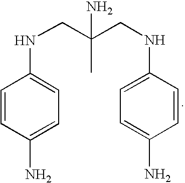

23. A multi-compartment device comprising at least one first compartment containing at least one dyeing composition, and at least one second compartment containing at least one oxidizing agent,
wherein the at least one dyeing composition comprises, in a medium suitable for dyeing, at least one para-phenylenediamine oxidation base chosen from compounds of formula (I) and the addition salts thereof: 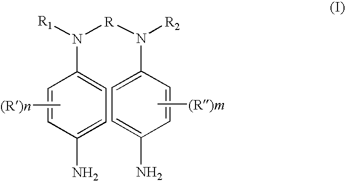
wherein:
R is chosen from:
linear or branched C₄-C₁₀ alkylene radicals substituted with at least one radical chosen from hydroxyl, C₁-C₄ alkoxy, amino, (C₁-C₄)monoalkylamino, and (C₁-C₄)dialkylamino radicals,
R₁ and R₂, which may be identical or different, are chosen from:
hydrogen,
linear or branched C₁-C₆ alkyl radicals, and
linear or branched C₁-C₆ alkyl radicals substituted with at least one radical chosen from hydroxyl, C₁-C₄ alkoxy, amino, (C₁-C₄)monoalkylamino, and (C₁-C₄)dialkylamino radicals,
R′ and R″, which may be identical or different, are chosen from:
C₁-C₆ alkyl radicals,
C₁-C₆ alkoxy radicals,
hydroxy(C₁-C₆)alkoxy radicals,
(C₁-C₆)alkoxy(C₁-C₆)alkyl radicals, and
mono- or polyhydroxy(C₁-C₆)alkyl radicals, and
n and m, which may be identical or different, are chosen from integers ranging from 0 to 4,
with the proviso that the at least one para-phenylenediamine oxidation base is not:

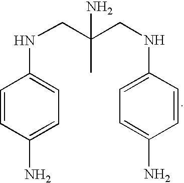

24. A process for the synthesis of a para-phenylenediamine compound of formula (I): 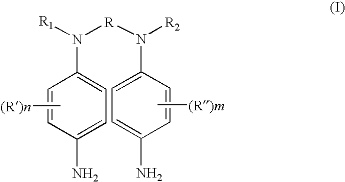
the process comprising reducing a nitrogen-containing compound of formula (II): 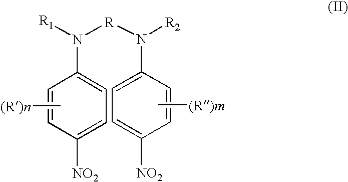
wherein in formulas (I) and (II):
R is chosen from:
linear or branched C₄-C₁₀ alkylene radicals substituted with at least one radical chosen from hydroxyl, C₁-C₄ alkoxy, amino, (C₁-C₄)monoalkylamino, and (C₁-C₄)dialkylamino radicals,
R₁ and R₂, which may be identical or different, are chosen from:
hydrogen,
linear or branched C₁-C₆ alkyl radicals, and
linear or branched C₁-C₆ alkyl radicals substituted with at least one radical chosen from hydroxyl, C₁-C₄ alkoxy, amino, (C₁-C₄)monoalkylamino, and (C₁-C₄)dialkylamino radicals,
R′ and R″, which may be identical or different, are chosen from:
C₁-C₆ alkyl radicals,
C₁-C₆ alkoxy radicals,
hydroxy(C₁-C₆)alkoxy radicals,
(C₁-C₆)alkoxy(C₁-C₆)alkyl radicals, and
mono- or polyhydroxy(C₁-C₆)alkyl radicals, and
n and m, which may be identical or different, are chosen from integers ranging from 0 to 4,
with the proviso that the para-phenylenediamine compound of formula (I) is not: 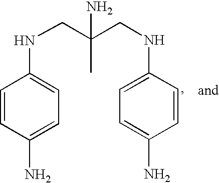
with the further proviso that the nitrogen-containing compound of formula (II) is not:

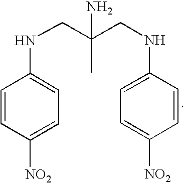

25. A nitrogen-containing compound of formula (II) 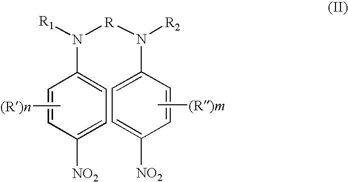
wherein:
R is chosen from:
linear or branched C₄-C₁₀ alkylene radicals substituted with at least one radical chosen from hydroxyl, C₁-C₄ alkoxy, amino, (C₁-C₄)monoalkylamino, and (C₁-C₄)dialkylamino radicals,
R₁ and R₂, which may be identical or different, are chosen from:
hydrogen,
linear or branched C₁-C₆ alkyl radicals, and
linear or branched C₁-C₆ alkyl radicals substituted with at least one radical chosen from hydroxyl, C₁-C₄ alkoxy, amino, (C₁-C₄)monoalkylamino, and (C₁-C₄)dialkylamino radicals,
R′ and R″, which may be identical or different, are chosen from:
C₁-C₆ alkyl radicals,
C₁-C₆ alkoxy radicals,
hydroxy(C₁-C₆)alkoxy radicals,
(C₁-C₆)alkoxy(C₁-C₆)alkyl radicals, and
mono- or polyhydroxy(C₁-C₆)alkyl radicals, and
n and m, which may be identical or different, are chosen from integers ranging from 0 to 4,
with the proviso that the nitrogen-containing compound of formula (II) is not: [chemical structure]
